(19) United States
(12) Patent Application Publication
(10) Pub. No.: US 2011/0027312 A1
(43) Pub. Date: Feb. 3, 2011

(54) CHIMERIC PORCINE CIRCOVIRUS PCV2Gen-1Rep AND USES THEREOF

(76) Inventors: Nicole M. Juhan, Morganton, NC (US); Xiang-Jin Meng, Blacksburg, VA (US)

Correspondence Address: ANNE M. ROSENBLUM, ESQ., 163 DELAWARE AVENUE, SUITE 212, DELMAR, NY, 12054, US

(21) Appl. No.: 12/937,181

(22) PCT Filed: Apr. 8, 2009
(86) PCT No.: PCT/US09/02189

§ 371 Date: Oct. 8, 2010

Related U.S. Application Data
(60) Provisional application No. 61/124,383, filed on Apr. 16, 2008.

(51) Int. Cl. A61K 39/12; C07H 21/04; C12N 15/63; C12N 5/10; C12N 7/00; C12P 21/00; A61K 39/00; A61P 31/20; A61P 37/04

(52) U.S. Cl. 424/204.1; 536/23.72; 435/320.1; 435/325; 435/235.1; 435/69.3; 424/184.1

(57) ABSTRACT

The present invention relates to a novel chimeric nucleic acid molecule of porcine circovirus (PCV2Gen-1Rep) that embraces a nucleic acid molecule encoding porcine circovirus type 2 (PCV2) which contains a nucleic acid sequence encoding a Rep protein of porcine circovirus type 1 (PCV1), particularly wherein the nucleic acid sequence encoding the Rep protein of PCV1 GO is an open reading frame (ORF) gene and, more particularly, wherein the ORF Rep gene is ORF1. A highly desirable chimeric nucleic acid molecule is constructed by replacing the ORF1 Rep gene of PCV2 by the ORF1 Rep gene of PCV1. The invention also encompasses the biologically functional plasmid or viral vector containing the unique chimeric nucleic acid molecules, suitable host cells transfected by the plasmid or vector, infectious chimeric porcine circoviruses that are produced by the suitable host cells, the process for the production of an immunogenic polypeptide product making use of the new chimera, viral vaccines that protect a pig against viral infection or postweaning multisystemic wasting syndrome (PMWS) caused by PCV2, methods of protecting a pig against viral infection or postweaning multisystemic wasting syndrome (PMWS) caused by PCV2, methods of preparing the unique chimera of PCV2Gen-1Rep and the like. This invention further includes a new method for improving the replication and titer of PCV2 in a cell culture.

### CROSS-REFERENCE TO RELATED U.S. APPLICATIONS

This application claims the benefit under 35 U.S.C. §119(e) of U.S. Provisional Application No. 61/124,383, filed on Apr. 16, 2008. The prior application is incorporated herein by reference in its entirety.

### STATEMENT REGARDING FEDERALLY SPONSORED RESEARCH OR DEVELOPMENT

Not Applicable

### REFERENCE TO A “SEQUENCE LISTING”

The material on a single compact disc containing a Sequence Listing file provided in this application is incorporated by reference.

### BACKGROUND OF THE INVENTION

1. Field of the Invention

The present invention concerns a unique chimeric porcine circovirus (PCV2Gen-1Rep) in which a nucleic acid sequence encoding a Rep protein of PCV1 is inserted into the genomic backbone of PCV2 and its use as an antigen in a new killed or attenuated chimera vaccine for the protection of pigs from viral infection or postweaning multisystemic wasting syndrome (PMWS) caused by PCV2.

2. Description of the Related Art

All patents and publications cited in this specification are hereby incorporated by reference in their entirety.

In 1974, porcine circovirus type 1 (PCV1) was originally isolated as a persistent contaminant of the PK-15 cell line ATCC CCL-33 (I. Tischer et al., “Characterization of papovavirus- and picornavirus-like particles in permanent pig kidney cell lines,” Zentralbl. Bakteriol. Hyg. Otg. A. 226(2):153-167 (1974)). Since its identification, PCV1 has been determined to be a ubiquitous swine virus that does not cause any disease in pigs (G. M. Allan et al., “Pathogenesis of porcine circovirus; experimental infections of colostrum deprived piglets and examination of pig foetal material,” Vet. Microbiol. 44:49-64 (1995); G. C. Dulac and A. Afshar, “Porcine circovirus antigens in PK-15 cell line (ATCC CCL-33) and evidence of antibodies to circovirus in Canadian pigs,” Can. J. Vet. Res. 53:431-433 (1989); S. Edwards and J. J. Sands, “Evidence of circovirus infection in British pigs,” Vet. Rec. 134:680-681 (1994); I. Tischer et al., “Studies on epidemiology and pathogenicity of porcine circovirus,” Arch. Virol. 91:271-276 (1986)). While PCV1 does not cause any disease in swine, it was subsequently determined that porcine circovirus type 2 is pathogenic. In 1991, the variant strain of PCV designated porcine circovirus type 2 (PCV2) was first recognized in pigs in Canada, and found to be associated with post-weaning multisystemic wasting syndrome (PMWS) (G. M. Allan et al., “Isolation of porcine circovirus-like viruses from pigs with a wasting disease in the USA and Europe,” J. Vet. Diagn. Invest. 10:3-10 (1998); I. Morozov et al., “Detection of a novel strain of porcine circovirus in pigs with postweaning multisystemic wasting syndrome,” J. Clin. Microbiol. 36:2535-2541 (1998)).

PCV1 and PCV2 have similar genomic organization with two main open reading frames (ORF): ORF1 encodes the viral Rep protein involved in virus replication and ORF2 encodes the viral capsid protein. Overall, PCV1 and PCV2 share 68-76% nucleotide sequence identity in their entire genome, while isolates within each genotype share greater than 90% identity (A. K. Cheung, “The essential and nonessential transcription units for viral protein synthesis and DNA replication of porcine circovirus type 2,” Virology 313:452-9 (2003)). PCV1 and PCV2 have similar genomic organization with two open reading frames (ORF) (A. K. Cheung, “Identification of the essential and non-essential transcription units for protein synthesis, DNA replication and infectious virus production of porcine circovirus type 1,” Arch. Virol. 149(5):975-88 (2004); A. K. Cheung, “Transcriptional analysis of porcine circovirus type 2,” Virology 305(1):168-180 (2003); A. Mankertz et al., “Identification of a protein essential for replication of porcine circovirus,” J. Gen. Virol. 79(Pt 2):381-384 (1998); P. Nawagitgul et al., “Open reading frame 2 of porcine circovirus type 2 encodes a major capsid protein,” J. Gen. Virol. 81:2281-2287 (2000)). In both viruses, ORF1 is responsible for viral replication and is alternatively spliced into 2 main functional proteins, Rep and Rep' (A. K. Cheung, “Identification of the essential and non-essential transcription units for protein synthesis, DNA replication and infectious virus production of porcine circovirus type 1,” Arch. Virol. 149(5):975-88 (2004); A. K. Cheung, “Transcriptional analysis of porcine circovirus type 2,” Virology 305(1):168-180 (2003); A. Mankertz and B. Hillenbrand, “Replication of porcine circovirus type 1 requires two proteins encoded by the viral rep gene,” Virology 279:429-38 (2001); A. Mankertz et al., “Identification of a protein essential for replication of porcine circovirus,” J. Gen. Virol. 79(Pt 2):381-384 (1998); A. Mankertz et al., “Mapping and characterization of the origin of DNA replication of porcine circovirus,” J. Virol. 71:2562-6 (1997)). ORF1 is highly conserved between PCV1 and PCV2 with approximately 83% nucleotide and 86% amino acid sequence identity (I. Morozov et al., “Detection of a novel strain of porcine circovirus in pigs with postweaning multisystemic wasting syndrome,” J. Clin. Microbiol. 36:2535-2541 (1998)). ORF2 encodes the immunogenic viral capsid protein in both viruses (P. Nawagitgul et al., “Open reading frame 2 of porcine circovirus type 2 encodes a major capsid protein,” J. Gen. Virol. 81:2281-2287 (2000)), and is more variable than the Rep protein with approximately 67% nucleotide and 65% amino acid sequence identity between PCV1 and PCV2 (I. Morozov et al., “Detection of a novel strain of porcine circovirus in pigs with postweaning multisystemic wasting syndrome,” J. Clin. Microbiol. 36:2535-2541 (1998)). Recently, a third ORF, ORF3, was identified in PCV2 but not in PCV1, and was reportedly involved in apoptosis (J. Liu et al., “Characterization of a previously unidentified viral protein in porcine circovirus type 2-infected cells and its role in virus-induced apoptosis,” J. Virol. 79:8262-74 (2005)).

Previously, U.S. Pat. No. 7,279,166 B2, U.S. Pat. No. 7,276,353 B2, M. Fenaux et al., “A chimeric porcine circovirus (PCV) with the immunogenic capsid gene of the pathogenic PCV type 2 (PCV2) cloned into the genomic backbone of the nonpathogenic PCV1 induces protective immunity against PCV2 infection in pigs,” J. Virol. 78:6297-303 (2004), and M. Fenaux et al., “Immunogenicity and pathogenicity of chimeric infectious DNA clones of pathogenic porcine circovirus type 2 (PCV2) and nonpathogenic PCV1 in weanling pigs,” J. Virol. 77:11232-243 (2003), have described the construction of a chimeric virus, designated PCV1-2, in which the ORF of PCV2 is cloned into the backbone of PCV1 genome. The publications highlight the interchange of the ORF2 capsid gene including its intergenic sequences from PCV2 in place of the ORF2 of PCV1 and further disclose an infectious reciprocal chimeric nucleic acid molecule of PCV2-1 comprising a nucleic acid molecule encoding PCV2 which has an immunogenic ORF2 gene from a nonpathogenic PCV1 in place of an ORF2 gene of the pathogenic PCV2 nucleic acid molecule. While the PCV1-2 chimera provided a naturally avirulent trait, the reciprocal chimeric nucleic acid molecule of PCV2-1, which was prepared merely as an experimental model, remained virulent without any commercial advantages over the parental PCV2 other than for research purposes to compare viral characteristics with PCV1-2. None of the aforementioned patents or articles discloses or suggests making any other reciprocal chimeric viruses utilizing the genomic backbone of the pathogenic PCV2, and certainly, none imply the exchange of alternative open reading frames beyond the specified and explicit ORF2 immunogenic capsid gene.

Further, the PCV1-2 chimeric virus replicates to titers similar to that of PCV2 in PK-15 cells (M. Fenaux et al., “Immunogenicity and pathogenicity of chimeric infectious DNA clones of pathogenic porcine circovirus type 2 (PCV2) and nonpathogenic PCV1 in weanling pigs,” J. Virol. 77:11232-243 (2003)). On the other hand, PCV1 has been adapted to grow better in PK-15 cells and the PK-15 cell culture-adapted PCV1 virus grows better than PCV2, replicating to at least approximately 1-log higher titer than PCV2 in PK-15 cells (M. Fenaux et al., “Two amino acid mutations in the capsid protein of type 2 porcine circovirus (PCV2) enhanced PCV2 replication in vitro and attenuated the virus in vivo,” J. Virol. 78:13440-6 (2004); M. Fenaux et al., “Immunogenicity and pathogenicity of chimeric infectious DNA clones of pathogenic porcine circovirus type 2 (PCV2) and nonpathogenic PCV1 in weanling pigs,” J. Virol. 77:11232-243 (2003)). This enhanced replication ability of PCV1 in PK-15 cells is likely due to the fact that PCV1 was originally isolated from the PK-15 cell line as a persistent cell culture contaminant, and thus is adapted to grow in PK-15 cells. However, the pathogenic PCV2 strains do not possess the same replication ability as PCV1, which poses a major problem associated with PCV2 vaccine production due to the relatively low titer of the pathogen in PK-15 cells. As a result, meeting efficient production levels of vaccines based upon PCV2, such as inactivated whole PCV2, naturally avirulent chimeric PCV1-2 (based on the genomic backbone of PCV1 origin), recombinant PCV2 capsid proteins and the like, is a real challenge and predicament to the vaccine manufacturers in the industry.

When the intergenic sequences of PCV2 were previously examined, an interferon-α-stimulated response element (ISRE)-like sequence (GAAANNGAAA) was identified in PCV2, which may activate gene transcription in response to IFN-α similar to the art-recognized activity of the ISRE-element (J. E. Darnell, Jr., et al., “Jak-STAT pathways and transcriptional activation in response to IFNs and other extracellular signaling proteins,” Science 264:1415-21 (1994)). It has been shown that Kaposi's sarcoma-associated herpesvirus expresses viral genes that interact with a viral encoded ISRE-like sequence which is responsible for additional viral gene activation (J. Zhang, “Kaposi's sarcoma-associated herpesvirus/human herpesvirus 8 replication and transcription activator regulates viral and cellular genes via interferon-stimulated response elements,” J. Virol. 79:5640-52 (2005)). Meerts et al. recently showed that both porcine kidney cells (PK-15) and porcine monocytic cells (3D4/31) treated with IFN-α after inoculation with PCV2 had an increased number of infected cells, up to 529% and 308%, respectively (P. Meerts et al., “Enhancement of porcine circovirus 2 replication in porcine cell lines by IFN-gamma before and after treatment and by IFN-alpha after treatment,” J. Interferon Cytokine Res 25(11):684-93

(November 2005)). Interestingly, PK-15 cells treated with IFN-α prior to inoculation with PCV2 had a decreased number of infected cells (69%) (id.). In addition to IFN-α, the effect of IFN-γ on PCV2 infection in both PK-15 and 3D4/31 cells have also been evaluated. It was found that treatment with IFN-γ after PCV2 infection resulted in a greater number of infected cells by 691% in PK-15 cells, and addition of IFN-γ before PCV2 inoculation increased the number of PCV2 antigen-positive cells by 706% in 3D4/31 cells, due to increased cellular internalization of the virus (id.). It is possible that a transcription factor responding to IFN-γ activation is present in the promoter region of the PCV2 sequence but at this point in time, it is all conjecture and not yet substantiated. Many viruses not only respond to, but also manipulate, IFN expression by a number of transcriptional pathways in order to circumvent cellular IFN response (S. Goodbourn, “Interferons: cell signalling, immune modulation, antiviral response and virus countermeasures,” J. Gen. Virol. 81:2341-64 (2000)). Taken together, the effect of IFN-α and IFN-γ on PCV2 infection in cell cultures and the role of the ISRE-like sequence in regulating IFN-α and IFN-γ responses remain to be studied. Nevertheless, adding interferon to stimulate the growth of PCV2 has had inconsistent results as shown by the prior research studies. While interferon can be given to pigs, there would be a concern about dosage level in an interferon-based final PCV2 product since interferon can cause adverse reactions and side effects particularly harmful to the liver. It would be more desirable to improve the replication properties of PCV2 by use of a natural component that can be safely administered to pigs.

Consequently, there is a definite art-recognized problem owing to insufficient quantities of antigen production during the manufacture of PCV2 vaccines that the present invention solves by developing a new porcine circovirus chimera that significantly improves the low titer and replication ability of the virus in order to manufacture larger batches of chimera vaccine than previously obtainable.

It is therefore an important object of the present invention to provide a unique combination of PCV1 and PCV2 that retains the antigenic ability of the pathogenic PCV2 to elicit adequate immune responses but innovatively attains the superior titer growth properties of PCV1.

It is a further important object of the present invention to make an improved chimera vaccine product based on PCV2 for protecting pigs against viral infection or postweaning multisystemic wasting syndrome (PMWS) caused by PCV2 wherein the improvement comprises better replication than the parental PCV2.

Further purposes and objects of the present invention will appear as the specification proceeds.

The foregoing objects are accomplished by providing a novel chimeric PCV2 virus containing the PCV1Rep gene in the genomic backbone of PCV2 as described herein.

### BRIEF SUMMARY OF THE INVENTION

The present invention concerns a unique chimeric porcine circovirus in which a nucleic acid sequence encoding the replication or Rep protein of porcine circovirus type 1 (PCV1) is incorporated into the genomic backbone of porcine circovirus type 2 (PCV2). A highly desirable embodiment of the invention relates to the construction of the PCV2Gen-1Rep chimera in which the open reading frame 1 (ORF1) Rep gene of PCV1 replaces the ORF1 Rep gene of PCV2 in the genomic structure of PCV2. The invention also encompasses the biologically functional plasmids, viral vectors and the like that contain the new chimeric nucleic acid molecules described herein, suitable host cells transfected by the plasmids or vectors comprising the chimera DNA and methods of preparing the chimeric constructs. Additionally included within the scope of the present invention are attenuated or inactivated vaccines comprising, for example, the chimeric DNA, a plasmid containing the chimeric DNA, a chimeric virus, etc. and novel methods of protecting pigs against viral infection or postweaning multisystemic wasting syndrome (PMWS) caused by PCV2 comprising administering to a pig in need of such protection an immunologically effective amount of the attenuated or inactivated vaccine. Surprisingly and advantageously, the chimeric porcine circovirus of this invention provides significantly improved replication and titers over the parental virus PCV2 and, thus, a further embodiment of the invention is drawn to a new method for improving the replication and titer of PCV2 in a cell culture.

### BRIEF DESCRIPTION OF THE DRAWINGS

The background of the invention and its departure from the art will be further described hereinbelow with reference to the accompanying drawings, wherein:

FIG. 1 shows that the chimeric SDM-C6 DNA clone (with the Rep gene of PCV1 cloned into the backbone of PCV2 genome) is infectious when transfected into PK-15 cells. Panels A and a illustrate PK-15 cells transfected with concatomerized SDM-C6 chimeric genome; Panels B and b illustrate PK-15 cells transfected with linearized single copy SDM-C6 chimeric genome; and Panels C and c illustrate transfection reagents and MEM as negative controls. Left panels provide the IFA results stained with a PCV2 ORF2 monoclonal antibody; while right panels provide PK-15 cell monolayers overlaid with the IFA results.

FIG. 2 illustrates the characterization of the growth characteristics of PCV1 (♦), PCV2 (▪), and chimeric SDM-C6 (Δ) viruses in PK-15 cells by one-step growth curve. PK-15 cells on 6-12 well plates were inoculated in duplicate with each virus at 0.1 multiplicity of infection. Two duplicate wells of infected cells were harvested every 12 hours, and the virus titers were determined by IFA.

### DETAILED DESCRIPTION OF THE INVENTION

In accord with the present invention, there are provided unique, infectious chimeric nucleic acid molecules of porcine circovirus (PCV2Gen-1Rep), live chimeric viruses produced from the chimeric nucleic acid molecule and veterinary vaccines to protect pigs from viral infection or postweaning multisystemic wasting syndrome (PMWS) caused by porcine circovirus type 2 (PCV2). The invention specifically deals with the construction and in vitro characterization of a chimeric nucleic acid molecule of porcine circovirus (PCV2Gen-1Rep) in which the nucleic acid molecule that encodes PCV2 contains a nucleic acid sequence encoding a replication (Rep) protein of porcine circovirus type 1 (PCV1). For purposes of inserting into the PCV2 nucleic acid molecule, the nucleic acid sequence encoding the Rep protein can be one or more functional nucleotide sequences that encode one or more replication proteins that are necessary for the viral replication of a non-pathogenic porcine circovirus strain. It is desirable to use a complete open reading frame (ORF) gene that encodes the Rep protein of PCV1 and, preferably, to use the ORF1 Rep gene of PCV1 for incorporation into the genomic backbone of PCV2. It is even more preferable for optimum chimera properties that the ORF1 Rep gene of PCV1 replaces an open reading frame of PCV2, particularly the ORF1 Rep gene of PCV2 in the genomic backbone of PCV2.

Another embodiment of the present invention involves a new method of preparing the chimeric nucleic acid molecule of PCV2Gen-1Rep as described herein, which comprises the following steps:

(a) removing a nucleic acid sequence that encodes a Rep protein from a nucleic acid molecule encoding PCV1;

(b) incorporating the nucleic acid sequence that encodes the Rep protein of PCV1 into a nucleic acid molecule encoding PCV2; and

(c) recovering the chimeric nucleic acid molecule.

The method can be conveniently modified to construct variations of the chimera virus of the present invention in which step (a) may involve removing the nucleic acid sequence comprising an open reading frame (ORF) gene that encodes the Rep protein of PCV1 or, more specifically, removing the nucleic acid sequence comprising the ORF1 Rep gene of PCV1. As an alternative embodiment of this invention, step (b) may further comprise removing the ORF1 gene of PCV2 and then incorporating the nucleic acid sequence comprising the ORF1 Rep gene of PCV1 into the ORF1 gene position of the nucleic acid molecule encoding PCV2.

The novel chimeric porcine circovirus of this invention is preferentially designed to provide significantly improved replication ability and enhanced titers over the parental virus PCV2. A representative chimera is constructed in the examples herein below and named “SDM-C6.” The SDM-C6 chimeric virus sample is infectious in vitro when transfected into PK-15 cells, indicating that the Rep gene between PCV1 and PCV2 are exchangeable.

The improved growth trait of the SDM-C6 chimeric virus is characterized by a one-step growth curve that compares the growth characteristics of the chimeric virus to the wild-type PCV1 and PCV2 viruses. The results demonstrate that the chimera virus surprisingly replicates at approximately 1-log titer higher and more efficiently than its parental virus PCV2 in cell cultures. Although the chimeric virus replicates to a similar titer as the non-pathogenic PCV1, the studies further show that the chimeric PCV2Gen-1Rep of the invention unexpectedly replicates more rapidly than both of its parental PCV1 and PCV2 viruses upon transfection (i.e., infection or inoculation) of PK-15 cells.

Since it has been problematic to grow PCV2 to a higher titer for adequate vaccine production on an industrial scale—where even a 1-log titer increase is considered significant—the present invention permits a remarkable advancement in the veterinary field that has important implications for PCV2 vaccine development. While a 1-log difference may not be significant for other viruses, the 1-log titer increase for PCV2 makes a huge difference in PCV2 vaccine production, that is, higher titers would reduce the vaccine volume per dose thereby increasing the potency of the final product and the efficiency of the entire manufacturing process. Advantageously, the use of the new PCV2Gen-1Rep chimera of the present invention provides for markedly better production of PCV2 vaccines than could previously be achieved in the past. Also, since the PCV2Gen-1Rep chimera is uniquely based on the genomic backbone of PCV2 origin, it makes an excellent antigenic substance in a vaccine for the protection of pigs against viral infection or PMWS caused by PCV2 owing to the presence, within the chimera construct, of the immunogenic ORF2 capsid gene of PCV2, which is important for eliciting an immune response in the inoculated pigs.

One of the major differences that distinguish the SDM-C6 chimeric virus of the present invention from the PCV1-2 chimeric virus described previously in U.S. Pat. No. 7,279,166 B2 and U.S. Pat. No. 7,276,353 B2 is the nucleic acid sequences: the genomic sequence of the PCV2Gen-1Rep chimeric virus of the present invention (SDM-C6) is of pathogenic PCV2 origin, whereas the previous PCV1-2 chimeric virus is of non-pathogenic PCV1 origin. It has been reported in the literature that the intergenic sequences between the Cap and Rep genes of the two viruses (PCV1 and PCV2) may play an important role in regulating PCV replication (A. K. Cheung, “Detection of rampant nucleotide reversion at the origin of DNA replication of porcine circovirus type 1,” Virology 333:22-30 (2005); A. K. Cheung, “Identification of an octanucleotide motif sequence essential for viral protein, DNA, and progeny virus biosynthesis at the origin of DNA replication of porcine circovirus type 2,” Virology 324:28-36 (2004); A. Mankertz et al., “New reporter gene-based replication assay reveals exchangeability of replication factors of porcine circovirus types 1 and 2,” J. Virol. 77:9885-93 (2003)). Since the alteration of a PCV2 strain in order to construct the chimeric SDM-C6 virus lies in the addition of a Rep gene from PCV1 in place of the Rep gene in the DNA sequence of PCV2, one may assume that the Rep gene of PCV1 might contribute to the enhanced replication ability. However, such assumptions would be pure speculation until the construct is actually made and tested in the growth studies particularly owing to the fact that non-pathogenic PCV1 and pathogenic PCV2 only share 68-76% nucleotide sequence homology in their entire genomes. The Rep gene of PCV1 may not translate to the functional codes of the PCV2 genome or provide better replication ability within the different nucleotide sequence encoding PCV2. Thus, the current observation in relation to the present invention that the chimeric SDM-C6 virus replicates to a similar titer as that of PCV1 is a surprising outcome. As another unexpected trait, the SDM-C6 virus also replicates faster than both PCV1 and PCV2 parental strains indicating that the mechanism of the enhanced replication ability for the PCV2Gen-1Rep chimeric virus of the present invention remains to be determined.

To date, there have been no previous reports of incorporating the Rep gene of PCV1 within the genomic backbone of pathogenic PCV2 and obtaining an infectious pathogen with improved replicating properties over both parental strains as described herein. In view of the enhanced replication ability of the new PCV2Gen-1Rep chimeric virus, a further embodiment of the present invention therefore relates to a novel method for improving the replication and titer of PCV2 in a cell culture which comprises the following steps:

(a) constructing a PCV2Gen-1Rep chimeric virus in which an ORF1 Rep gene of PCV2 is replaced by the ORF1 Rep gene of PCV1;

(b) inoculating a suitable cell line with the PCV2Gen-1Rep chimera;

(c) culturing the PCV2Gen-1Rep chimera in a suitable virus growth medium under standard conditions for a sufficient amount of time to induce virus production; and

(d) harvesting the chimera virus.

In the foregoing method, examples of a suitable cell line would be a porcine kidney cell line free of porcine antigen (PK-15 cells), a swine testicle (ST) cell line and similar cell lines capable of growing porcine circoviruses or specifically adapted for growing porcine circoviruses.

Also included within the scope of the present invention are biologically functional plasmids, viral vectors and the like that contain the new chimeric nucleic acid molecules described herein, suitable host cells transfected by those plasmids or vectors and live chimeric porcine circovirus produced by the host cells. The invention further embraces a process for the production of an immunogenic polypeptide product in which the process comprises growing, under suitable nutrient conditions, prokaryotic or eucaryotic host cells transfected with the chimeric nucleic acid molecule of porcine circovirus (PCV2Gen-1Rep), as described herein, in a manner allowing expression of said polypeptide product, and isolating the desired polypeptide product of the expression of the chimeric molecule.

Suitably attenuated or inactivated (i.e., killed) vaccines of the chimeric viral and DNA molecules, and methods of using them, are also included within the scope of the present invention. Inoculated pigs are protected from serious viral infection and PMWS caused by PCV2. The novel method protects pigs in need of protection against viral infection or PMWS by administering to the pig an immunologically effective amount of a vaccine according to the invention, such as, for example, a vaccine comprising an immunogenic amount of the chimeric DNA sequence encoding PCV2Gen-1Rep, the cloned chimera virus, a plasmid or viral vector containing the chimeric DNA molecules, the recombinant PCV2Gen-1Rep DNA sequence, etc. Other antigens such as PRRSV, PPV, other infectious swine agents and immune stimulants may be given concurrently to the pig to provide a broad spectrum of protection against viral infections.

The vaccines comprise, for example, the chimeric nucleic acid molecule of PCV2Gen-1 Rep, the cloned chimeric genome in suitable plasmids or vectors such as, for example, the pSK vector, a killed (inactivated) or attenuated chimeric virus, etc. in combination with a nontoxic, physiologically acceptable carrier and, optionally, one or more standard adjuvants. Preferably, the vaccine uses a killed chimera virus as the antigen.

The adjuvant, which may be administered in conjunction with the vaccine of the present invention, is a substance that increases the immunological response of the pig to the vaccine. The adjuvant may be administered at the same time and at the same site as the vaccine, or at a different time, for example, as a booster. Adjuvants also may advantageously be administered to the pig in a manner or at a site different from the manner or site in which the vaccine is administered. Suitable adjuvants known to those of ordinary skill in the veterinary field include, but are not limited to, aluminum hydroxide (alum), immunostimulating complexes (ISCOMS), non-ionic block polymers or copolymers, cytokines (like IL-1, IL-2, IL-7, IFN-α, IFN-β, IFN-γ, etc.), saponins, monophosphoryl lipid A (MLA), muramyl dipeptides (MDP) and the like. Other suitable adjuvants include, for example, aluminum potassium sulfate, heat-labile or heat-stable enterotoxin isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde.

The vaccines may further contain additional antigens to promote the immunological activity of the chimeric virus or DNA of the present invention such as, for example, porcine reproductive and respiratory syndrome virus (PRRSV), porcine parvovirus (PPV), other infectious swine agents and immune stimulants.

The new vaccines of this invention are not restricted to any particular type or method of preparation. The cloned viral vaccines include, but are not limited to, infectious DNA vaccines (i.e., using plasmids, vectors or other conventional carriers to directly inject DNA into pigs), attenuated vaccines, inactivated (killed) vaccines, genetically engineered vaccines, etc. These vaccines are prepared by standard methods known in the art.

Since the antigenic substance in the vaccine of this invention is based on the pathogenic PCV2 strain, the active agent must first be attenuated or inactivated by a suitable art-recognized method. To prepare inactivated virus vaccines, for instance, the virus propagation from the infectious DNA clone is done by methods known in the art or described herein. Serial virus inactivation is then optimized by protocols generally known to those of ordinary skill in the art.

Inactivated virus vaccines may be prepared by treating the chimeric virus derived from the cloned DNA with inactivating agents such as formalin or hydrophobic solvents, acids, etc., by irradiation with ultraviolet light or X-rays, by heating, etc. Inactivation is conducted in a manner understood in the art. For example, in chemical inactivation, a suitable virus sample or serum sample containing the virus is usually treated for a sufficient length of time with a sufficient amount or concentration of inactivating agent at a sufficiently high (or low, depending on the inactivating agent) temperature or pH to inactivate the virus. Inactivation by heating is typically conducted at a temperature and for a length of time sufficient to inactivate the virus. Inactivation by irradiation is often conducted using a wavelength of light or other energy source for a length of time sufficient to inactivate the virus. Generally, the terms “inactivated,” “dead” or “killed” are used interchangeably in the context of viral vaccines to mean the vaccine contains viruses that have been inactivated. The virus is considered inactivated if it is unable to infect a cell susceptible to infection.

To prepare attenuated vaccines from pathogenic clones, the tissue culture adapted, live, pathogenic PCV2Gen-1Rep is first attenuated (rendered nonpathogenic or harmless) by methods known in the art, typically made by serial passage through cell cultures. Attenuation of pathogenic clones may also be made by gene deletions or viral-producing gene mutations.

It is further possible to pinpoint the nucleotide sequences in the viral genome responsible for virulence, and genetically engineer the virus avirulent through, for example, site-directed mutagenesis. Site-directed mutagenesis is able to add, delete or change one or more nucleotides (see, for instance, Zoller et al., DNA 3:479-488, 1984). An oligonucleotide is synthesized containing the desired mutation and annealed to a portion of single stranded viral DNA. The hybrid molecule, which results from that procedure, is employed to transform bacteria. Then double-stranded DNA, which is isolated containing the appropriate mutation, is used to produce full-length DNA by ligation to a restriction fragment of the latter that is subsequently transfected into a suitable cell culture. Ligation of the genome into the suitable vector for transfer may be accomplished through any standard technique known to those of ordinary skill in the art. Transfection of the vector into host cells for the production of viral progeny may be done using any of the conventional methods such as calcium-phosphate or DEAE-dextran mediated transfection, electroporation, protoplast fusion and other well-known techniques (e.g., Sambrook et al., “Molecular Cloning: A Laboratory Manual,” Cold Spring Harbor Laboratory Press, 1989). The cloned virus then exhibits the desired mutation. Alternatively, two oligonucleotides can be synthesized which contain the appropriate mutation. These may be annealed to form double-stranded DNA that can be inserted in the viral DNA to produce full-length DNA.

An insect cell line (like HI-FIVE) can be transformed with a transfer vector containing nucleic acid molecules obtained from the virus or copied from the viral genome which encodes one or more of the immuno-dominant proteins of the virus. The transfer vector includes, for example, linearized baculovirus DNA and a plasmid containing the immunogenic polynucleotides. The host cell line may be co-transfected with the linearized baculovirus DNA and a plasmid in order to make a recombinant baculovirus. Alternatively, live vectors, such as a poxvirus or an adenovirus, can be used as a vaccine in combination with the chimera of the invention.

An immunologically effective amount of the vaccines of the present invention is administered to a pig in need of protection against viral infection or PMWS. The immunologically effective amount or the immunogenic amount that inoculates the pig can be easily determined or readily titrated by routine testing. An effective amount is one in which a sufficient immunological response to the vaccine is attained to protect the pig exposed to the virus which causes PMWS. Preferably, the pig is protected to an extent in which one to all of the adverse physiological symptoms or effects of the viral disease are significantly reduced, ameliorated or totally prevented.

The vaccine can be administered in a single dose or in repeated doses. Dosages may range, for example, from about 1 microgram to about 1,000 micrograms of the plasmid DNA containing the infectious chimeric DNA genome (dependent upon the concentration of the immuno-active component of the vaccine), preferably 100 to 200 micrograms of the chimeric PCV2Gen-1Rep DNA clone. Methods are known in the art for determining or titrating suitable dosages of active antigenic agent to find minimal effective dosages based on the weight of the pig, concentration of the antigen and other typical factors.

Desirably, the vaccine is administered to a pig not yet exposed to the PCV virus. The vaccine containing the antigenic substance can conveniently be administered intranasally, transdermally (i.e., applied on or at the skin surface for systemic absorption), parenterally, etc. The parenteral route of administration includes, but is not limited to, intramuscular, intravenous, intraperitoneal, intradermal (i.e., injected or otherwise placed under the skin), subcutaneous routes and the like. Since the intramuscular and intradermal routes of inoculation have been successful in other studies using viral infectious DNA clones (E. E. Sparger et al., “Infection of cats by injection with DNA of feline immunodeficiency virus molecular clone,” Virology 238:157-160 (1997); L. Willems et al., “In vivo transfection of bovine leukemia provirus into sheep,” Virology 189:775-777 (1992)), these routes are most preferred, in addition to the practical intranasal route of administration. Although less convenient, it is also contemplated that the vaccine is given to the pig through the intralymphoid route of inoculation.

When administered as a liquid, the present vaccine may be prepared in the form of an aqueous solution, syrup, an elixir, a tincture and the like. Such formulations are known in the art and are typically prepared by dissolution of the antigen and other typical additives in the appropriate carrier or solvent systems. Suitable carriers or solvents include, but are not limited to, water, saline, ethanol, ethylene glycol, glycerol, etc. Typical additives are, for example, certified dyes, flavors, sweeteners and antimicrobial preservatives such as thimerosal (sodium ethylmercurithiosalicylate). Such solutions may be stabilized, for example, by addition of partially hydrolyzed gelatin, sorbitol or cell culture medium, and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Liquid formulations also may include suspensions and emulsions that contain suspending or emulsifying agents in combination with other standard co-formulants. These types of liquid formulations may be prepared by conventional methods. Suspensions, for example, may be prepared using a colloid mill. Emulsions, for example, may be prepared using a homogenizer.

Parenteral formulations, designed for injection into body fluid systems, require proper isotonicity and pH buffering to the corresponding levels of porcine body fluids. Isotonicity can be appropriately adjusted with sodium chloride and other salts as needed. Suitable solvents, such as ethanol or propylene glycol, can be used to increase the solubility of the ingredients in the formulation and the stability of the liquid preparation. Further additives that can be employed in the present vaccine include, but are not limited to, dextrose, conventional antioxidants and conventional chelating agents such as ethylenediamine tetraacetic acid (EDTA). Parenteral dosage forms must also be sterilized prior to use.

The following examples demonstrate certain aspects of the present invention. However, it is to be understood that these examples are for illustration only and do not purport to be wholly definitive as to conditions and scope of this invention. It should be appreciated that when typical reaction conditions (e.g., temperature, reaction times, etc.) have been given, the conditions both above and below the specified ranges can also be used, though generally less conveniently. The examples are conducted at room temperature (about 23° C. to about 28° C.) and at atmospheric pressure. All parts and percents referred to herein are on a weight basis and all temperatures are expressed in degrees centigrade unless otherwise specified.

A further understanding of the invention may be obtained from the non-limiting examples that follow below.

### Example 1

### Construction of Chimeric PCV2Gen-1Rep

Throughout the in vitro experiments that follow below, PCV-free PK-15 cells were used. These cells were previously derived by end-point dilution (M. Fenaux et al., “Cloned genomic DNA of type 2 porcine circovirus is infectious when injected directly into the liver and lymph nodes of pigs: characterization of clinical disease, virus distribution, and pathologic lesions,” J. Virol. 76:541-51 (2002)). Constructions of the PCV2 and PCV1 single copy and dimerized tandem repeat infectious DNA clones were previously described (id.).

To construct the chimeric PCV2Gen-1Rep with the ORF1 Rep gene of PCV1 replacing that of PCV2 in the backbone of the PCV2 genome (including its intergenic sequences), the single copy genome of PCV1 infectious DNA clone in pBluescript II SK+ vector was amplified by PCR with primers PCV1REPF (5′ CAACTGGCCAAGCAAGAAAAG 3′ (which corresponds to SEQ ID NO:1)) and PCV1REPR (5′ AACCATTACGATGTGATCAAAAAGACTCAGTAAT TTATTTTATATGGGAAAAGGG 3′ (which corresponds to SEQ ID NO:2)) to produce the PCV1Rep gene fragment with engineered restriction enzyme sites BalI and BclI at either end. The PCR reaction consisted of 45 μl of Platinum PCR SuperMix High Fidelity (Invitrogen, Carlsbad, Calif.), 20 μM of primer PCV1 REPR, 20 μM of primer PCV1 REPF, and 1 μl of the single copy PCV1 infectious DNA clone. The thermocycler reaction consisted of an initial denaturation for 2 min at 94° C., and 35 cycles of denaturation at 94° C. for 30 s, annealing at 55° C. for 30 s, and extension at 68° C. for 30 s, followed by a final incubation at 68° C. for 7 min. The PCV1Rep fragment was separated by 1% agarose gel, and purified using the Geneclean II Kit (Qbiogene, Irvine, Calif.). The PCV1Rep fragment was then digested with BalI and BclI, separately, and the resulting digested fragment was run in a 1% agarose gel and purified using the Geneclean II Kit.

The PCV2 genomic backbone fragment minus the Rep gene was amplified from the single copy PCV2 infectious DNA clone in pBluescript vector by PCR using primers PCV2GENF (5′ CTTTTTGATCACTTCGTAATGGTTTTTA 3′ (which corresponds to SEQ ID NO:3)) and PCV2GENR (5′ GCTTACCATGTTGCTGCTGAGGT 3′ (which corresponds to SEQ ID NO:4)). The BfrBI and BclI restriction enzyme sites were introduced at either end of the fragment. The PCR reaction consisted of 20 pM of primer PCV2GENF, 20 μM of primer PCV2GENR, 40 mM dNTP (Fisher Scientific, Pittsburgh, Pa.), 200 mM MgCl₂, 10 μl 10×PCR buffer, 72 μl dH2O, 5 units AmpliTaq (Applied Biosystems, Foster City, Calif.), and 1 μl of the single copy PCV2 infectious DNA clone. The thermocycler reaction consisted of an initial denaturation at 94° C. for 10 min, and 38 cycles of denaturation at 94° C. for 1 min, annealing at 50° C. for 1 min, and extension at 72° C. for 45 s, followed by a final extension at 72° C. for 7 min. The PCV2 genomic backbone fragment (without Rep gene), PCV2Gen fragment, was separated by 1% agarose gel and purified using the Geneclean II Kit. The PCV2Gen fragment was then digested with BfrBI and BclI, separately, run on a 1% agarose gel, and purified using the Geneclean II Kit.

To generate the chimeric PCV infectious DNA clone, overnight ligation of the PCV1Rep and PCV2Gen fragments was performed using the Stratagene DNA ligation kit (LaJolla, Calif.). The ligation mixture was used to transform TOP10 cells (Invitrogen) according to the manufacturer's protocol. White colonies were selected, cultured overnight, and the plasmids were extracted using Sigma's GenElute Plasmid Miniprep Kit (St. Louis, Mo.). The plasmids were digested with the restriction enzyme KpnI and run on a 1% agarose gel to identify authentic plasmids with 2 bands of approximately 1.7 kb (PCV2Gen-1Rep) and 2.9 kb (pBluescript II SK+ vector).

### Example 2

### Viability Testing of Chimeric PCV2Gen-1Rep DNA Clone

Viability testing of the chimeric PCV2Gen-1Rep DNA clone was performed by transfection of PK-15 cells. The restriction enzyme KpnI was used to excise the chimeric PCV2Gen-1Rep genome from the pBluescript II SK+ plasmid vector. The chimeric PCV2Gen-1Rep genome was run on a 1% agarose gel, purified using GeneClean II, and subsequently concatomerized with T4 DNA ligase, essentially using conventional techniques previously described (M. Fenaux et al., 2002, supra). PK-15 cells at approximately 70% confluency growing on Lab-Tek chamber slide were transfected with concatomerized PCV2Gen-1Rep genome DNA using Lipofectamine and Plus Reagent according to the manufacturer's protocol (Invitrogen). Three days after transfection, indirect immunofluorescence assay (IFA) using a PCV2 ORF2-specific polyclonal antibody was performed as previously described (id.) to determine the infectivity. To further assess the infectivity of the PCV2Gen-1Rep chimeric genome, PK-15 cells at 70% confluency growing in T-25 flasks were transfected with approximately 12 μg of concatomerized chimeric genome per flask as previously described (id.). Virus stock was harvested 3 days after transfection and titrated by IFA with a PCV2 ORF2-specific polyclonal antibody as previously described (id.).

### Example 3

### DNA Sequencing To Confirm Chimeric Genome

### Primers Rep830F (5′ GGTGTCTTCTTCTGCGGTAACG 3′ (which corresponds to SEQ ID NO:5)) and Rep830R (5′ GTTCTACCCTCTTCCAAACCTTCC 3′ (which corresponds to SEQ ID NO:6)) were used to amplify the junction region between the 3′ of the PCV2Gen fragment and the 5′ of the PCV1Rep fragment. Primers Rep10F (5′ GGAAGACTGCTGGAGAACAATCC 3′ (which corresponds to SEQ ID NO:7)) and Rep1 OR (5′ CGTTACTTCACACCCAAACCTG 3′ (which corresponds to SEQ ID NO:8)) were used to amplify the junction region between the 5′ of the PCV1Rep fragment and the 3′ of the PCV2Gen fragment. The amplified PCR products were sequenced for both strands.

### Example 4

### Site-Directed Mutagenesis

The initial chimeric PCV2Gen-1Rep DNA clone was not infectious when transfected into PK-15 cells. After analyzing the sequence of the chimeric genome, a 6 nucleotide (GTAAGC) insertion was identified after the ATG start codon of the PCV10RF1 Rep gene. To correct this error and unwanted insertion introduced through the PCR and the cloning steps, primers MVTF (5′ CTCAGCAGCAACATGCCAAGCAAGAAAAGCGG 3′ (which corresponds to SEQ ID NO:9)) and MVTR (5′ CCGCTTTTCTTGCTTGGCATGTTGC TGCTGAG 3′ (which corresponds to SEQ ID NO:10)) were used to delete the 6 nucleotide insertion using the QuikChange II Site-Directed Mutagenesis Kit (Stratagene). TOP10 cells were transformed with the mutagenized product according to the manufacturer's protocol (Invitrogen). White colonies were selected and cultured overnight. Clone SDM-C6 was streaked on an LB agar plate containing ampicillin and grown overnight at 37° C. Four colonies were selected and cultured overnight. Their plasmids were extracted and sequenced using primers Rep830F and Rep830R to ensure that the introduced 6 nucleotides were removed from the chimeric genome.

It was found that the 6 nucleotide (GTAAGC) insertion rendered the chimeric clone non-infectious and the unwanted insertion was successfully removed by site-directed mutagenesis. The new chimeric clone, SDM-C6, was found to be infectious upon subsequent transfection into PK-15 cells.

### Example 5

### Preparation of Virus Stocks for In Vitro Characterization of Chimeric Virus

PCV1 and PCV2 virus stocks were prepared, respectively, from PCV1 and PCV2 infectious DNA clones by transfection of PK-15 cells in accordance with conventional techniques previously described (M. Fenaux et al., 2002, supra). The infectious titer of each virus stock was determined by IFA with a PCV2 ORF2-specific antibody (id.).

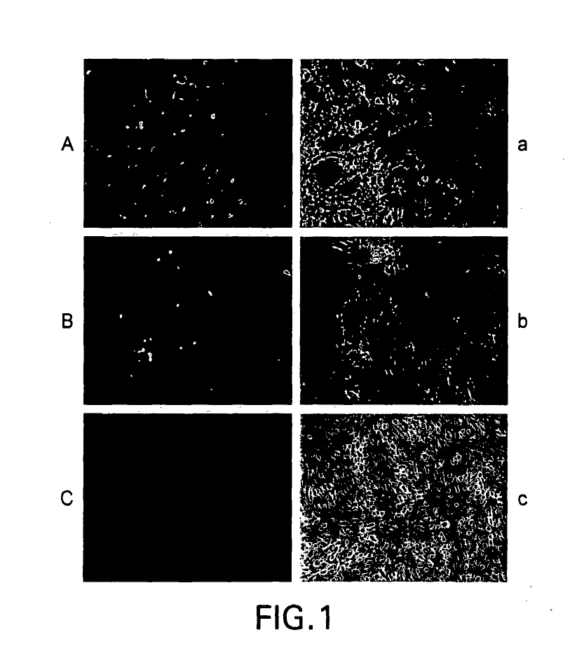

The SDM-C6 chimeric genome containing PCV1Rep gene in the backbone of PCV2 genome was excised from the pBluescript II SK+ plasmid using the restriction enzyme KpnI, and purified using the GeneClean II kit. Approximately 40 μg of the SDM-C6 chimeric genome was concatomerized with T4 DNA ligase and used to transfect 4 flasks (10 μg per flask) of PK-15 cells at approximately 70% confluency using Lipofectamine and Plus Reagent as previously described (id.). Three days post-transfection, the SDM-C6 chimeric virus was harvested by freezing and thawing the transfected cells three times, and the infectious titer of the chimeric SDM-C6 virus stock was determined by IFA with a PCV2 ORF2 monoclonal antibody (Rural Technologies Inc., Brookings, S. Dak.) at a dilution of 1:1000 in Phosphate-Buffered Saline (10×, pH 7.4) (Invitrogen). The SDM-C6 virus stock had an excellent virus infectious titer of 0.5×10^{5.5} TCID₅₀/ml. PK-15 cells transfected with both concatomerized and linearized SDM-C6 genome were strongly positive by IFA (FIG. 1) establishing that the SDM-C6 chimeric genome with PCV1Rep gene cloned in the backbone of the PCV2 genome is infectious in vitro.

### Example 6

### One-Step Growth Curve

To characterize the growth characteristics of the chimeric virus, and compare it to the wild-type PCV1 and PCV2 viruses, a one-step growth curve was performed. PK-15 cells were cultured in eight wells of six 12-well plates. At approximately 70% confluency, each well was washed with 2 mL of MEM. Eight wells in duplicate plates were each inoculated with PCV1, PCV2, and SDM-C6 at 0.1 multiplicity of infection (MOI). After 1 hour incubation, the inoculum was removed. The cell monolayers were subsequently washed three times each with 2 mL of PBS to remove any excess amount of virus inoculum. Two mL of MEM with 2% FBS and 1× antibiotic-antimycotic was added to each well, and the plates were continuously incubated at 37° C. with 5% CO₂. At 0, 12, 24, 36, 48, 60, 72, 84, and 96 hours post-inoculation (hpi), the cells in one well of each duplicate plate were harvested by scraping into the supernatant. The harvested cells were frozen and thawed 3 times and stored at −80° C. until titration. The infectious titer at each hpi was determined in 8-well Lab-Tek II chamber slides (Nalge Nunc International, Rochester, N.Y.) using serially diluted inocula followed by IFA with a PCV2 ORF2 monoclonal antibody using the Spearman-Karber method (M. Fenaux et al., 2002, supra).

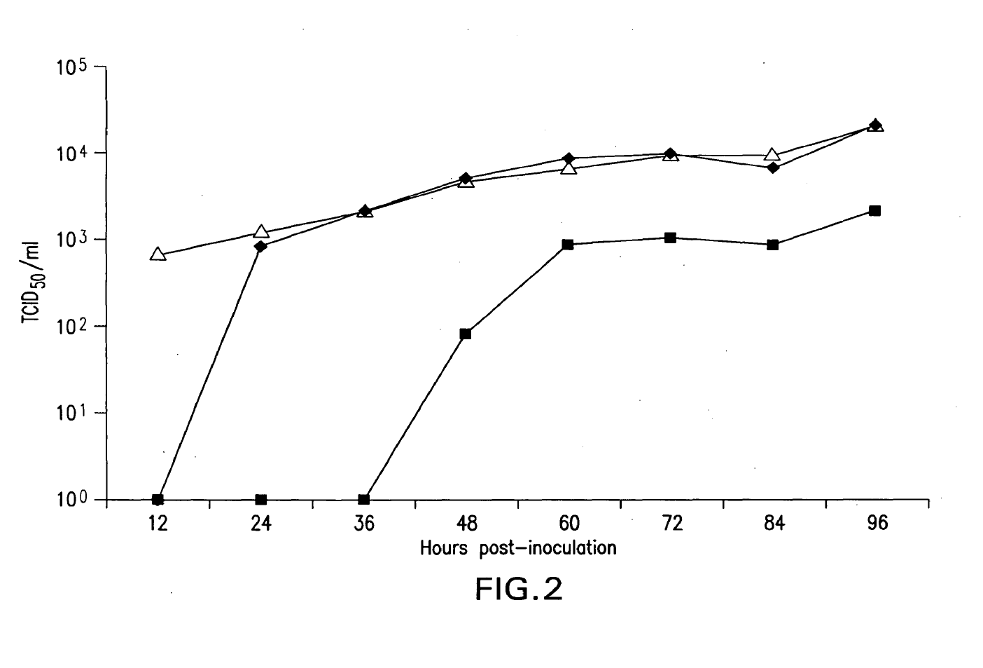

The data showed that the SDM-C6 chimeric virus grew as well as the PCV1 virus (FIG. 2) grew to a titer of 2.20×10^{4.0} TCID₅₀/mL at 96 hpi, whereas PCV2 grew to only 2.20×10^{3.0} TCID₅₀/mL at 96 hpi. At 12 hpi, the chimeric SDM-C6 virus grew to a titer of 6.95×10^{2.0} TCID₅₀/mL, whereas the PCV1 and PCV2 viruses both had undetectable titers, permitting the conclusion that the chimeric virus replicates faster than the parental viruses. PCV1 had a detectable virus titer of 8.70×10^{2.0} TCID₅₀/mL by 24 hpi, whereas PCV2 did not have a detectable titer until 48 hpi (7.91×10^{1.0} TCID₅₀/mL). Hence, it was observed that the chimeric SDM-C6 virus and PCV1 virus grew to similar titers, which was approximately 1-log higher than that of the parental PCV2 virus.

In the foregoing, there has been provided a detailed description of particular embodiments of the present invention for the purpose of illustration and not limitation. It is to be understood that all other modifications, ramifications and equivalents obvious to those having skill in the art based on this disclosure are intended to be included within the scope of the invention as claimed.

What is claimed is:

1. A chimeric nucleic acid molecule of porcine circovirus (PCV2Gen-1Rep) comprising a nucleic acid molecule encoding porcine circovirus type 2 (PCV2) which contains a nucleic acid sequence encoding a Rep protein of porcine circovirus type 1 (PCV1).

2. The chimeric nucleic acid molecule according to claim 1, wherein the nucleic acid sequence encoding the Rep protein of PCV1 is an open reading frame (ORF) gene.

3. The chimeric nucleic acid molecule according to claim 2, wherein the ORF Rep gene is ORF1.

4. The chimeric nucleic acid molecule according to claim 3, wherein the ORF1 Rep gene of PCV2 is replaced by the ORF I Rep gene of PCV1.

5. A biologically functional plasmid or viral vector containing the chimeric nucleic acid molecule according to any one of claims 1 to 4.

6. A suitable host cell transfected by the plasmid or vector according to claim 5.

7. An infectious chimeric porcine circovirus produced by host cells according to claim 6.

8. A process for the production of an immunogenic polypeptide product, said process comprising: growing, under suitable nutrient conditions, prokaryotic or eucaryotic host cells transfected with the chimeric nucleic acid molecule of porcine circovirus according to any one of claims 1 to 4 in a manner allowing expression of said polypeptide product, and isolating the desired polypeptide product of the expression of the chimeric molecule.

9. A viral vaccine that protects a pig against viral infection or postweaning multisystemic wasting syndrome (PMWS) caused by PCV2 comprising a nontoxic, physiologically acceptable carrier and an immunogenic amount of a suitably attenuated or inactivated member selected from the group consisting of:
(a) a chimeric nucleic acid molecule of porcine circovirus (PCV2Gen-1Rep) comprising a nucleic acid molecule encoding porcine circovirus type 2 (PCV2) which contains a nucleic acid sequence encoding a Rep protein of porcine circovirus type 1 (PCV1);
(b) a biologically functional plasmid or viral vector containing a chimeric nucleic acid molecule of porcine circovirus (PCV2Gen-1Rep) comprising a nucleic acid molecule encoding PCV2 which contains a nucleic acid sequence encoding a Rep protein of porcine circovirus type 1 (PCV1); and
(c) an infectious chimeric porcine circovirus made from a chimeric nucleic acid molecule of porcine circovirus (PCV2Gen-1Rep) comprising a nucleic acid molecule encoding PCV2 which contains a nucleic acid sequence encoding a Rep protein of porcine circovirus type 1 (PCV1).

10. The vaccine of claim 9, wherein the chimeric nucleic acid molecule contains a nucleic acid sequence encoding the Rep protein of PCV1 that comprises an open reading frame (ORF) gene.

11. The vaccine of claim 10, wherein the chimeric nucleic acid molecule contains the ORF1 Rep gene of PCV1.

12. The vaccine of claim 11, wherein the chimeric nucleic acid molecule contains the ORF1 Rep gene of PCV1 in place of the ORF1 Rep gene of PCV2.

13. A method of protecting a pig against viral infection or postweaning multisystemic wasting syndrome (PMWS) caused by PCV2 comprising administering to the pig in need of protection an immunologically effective amount of the vaccine according to any one of claims 9 to 12.

14. The method according to claim 13, which comprises administering the vaccine parenterally, intranasally, intradermally or transdermally to the pig.

15. A method of preparing the chimeric nucleic acid molecule of PCV2Gen-1Rep according to claim 1, which comprises the following steps:
(a) removing a nucleic acid sequence that encodes a Rep protein from a nucleic acid molecule encoding PCV1;
(b) incorporating the nucleic acid sequence that encodes the Rep protein of PCV1 into a nucleic acid molecule encoding PCV2; and
(c) recovering the chimeric nucleic acid molecule.

16. The method according to claim 15, wherein step (a) involves removing the nucleic acid sequence comprising an open reading frame (ORF) gene that encodes the Rep protein of PCV1.

17. The method according to claim 16, wherein step (a) involves removing the nucleic acid sequence comprising the ORF1 Rep gene of PCV1.

18. The method according to claim 17, wherein step (b) further comprises removing the ORF1 gene of PCV2 and then incorporating the nucleic acid sequence comprising the ORF1 Rep gene of PCV1 into the ORF1 gene position of the nucleic acid molecule encoding PCV2.

19. A method for improving the replication and titer of PCV2 in a cell culture which comprises the following steps:
(a) constructing a PCV2Gen-1Rep chimeric virus in which an ORF1 Rep gene of PCV2 is replaced by the ORF1 Rep gene of PCV1;
(b) inoculating a suitable cell line with the PCV2Gen-1Rep chimera;
(c) culturing the PCV2Gen-1Rep chimera in a suitable virus growth medium under standard conditions for a sufficient amount of time to induce virus production; and
(d) harvesting the chimera virus.

20. The method according to claim 19, wherein the suitable cell line is a porcine kidney cell line free of porcine antigen (PK-15 cells) or a swine testicle (ST) cell line.
